(19) United States
(12) Patent Application Publication
(10) Pub. No.: US 2007/0299316 A1
(43) Pub. Date: Dec. 27, 2007

(54) SYSTEM AND METHOD FOR REMOTE MEDICAL DEVICE OPERATION

(76) Inventors: Patrick Haslehurst, (US); Jeremy Brouillette, (US)

Correspondence Address: BERESKIN AND PARR, 40 KING STREET WEST, BOX 401, TORONTO, ON, M5H 3Y2, US

(21) Appl. No.: 11/425,626

(22) Filed: Jun. 21, 2006

(51) Int. Cl. A61B 5/00

(52) U.S. Cl. 600/300

(57) ABSTRACT

A method for conducting examinations of patients at remote locations. The method comprises establishing a medical session between a primary physician and a patient; instructing during the medical session the patient from a physician station through a communication channel to engage one or more medical devices associated with a patient station; generating a request during the medical session for the one or more medical devices to take a reading from the patient that generates reading data and to transmit the reading data from the one or more medical devices to the physician station; and receiving at the physician station during the medical session the reading data from the one or more medical devices.

### FIELD OF THE INVENTION

The invention relates generally to a system and method for conducting examinations of patients in remote locations.

### BACKGROUND OF THE INVENTION

When examining a patient in person, a physician has at their disposal all of the required amenities that are found in a physician's office. By having such amenities, including a multitude of medical devices that may be used to conduct an examination upon a patient, the patient may be thoroughly examined. Additionally, the physician is able to visually observe the patient, and pay specific attention to any particular areas upon the patient's body that require further attention.

The physician will keep notes detailing the appointment, and any observations made by the physician. The notes also will include any readings taken from the medical devices. While direct contact between physician and patient is often desired, it is often difficult for individuals to be able to visit a physician due to location, time, and other such factors. As many individuals are unable to visit with physicians in person on a regular basis, much research has been conducted into systems that may be referred to as telemedicine systems.

Telemedicine systems generally allow a physician to interact with a patient in a remote location. Conventional telemedicine systems allow the physician to observe and interact the patient, and discuss any ailments that may plague the patient. Conventional telemedicine systems however do not provide the physician with all of the amenities they have associated with their respective offices.

### SUMMARY OF THE INVENTION

In accordance with a first aspect of the invention, there is provided a method for conducting examinations of patients at remote locations. The method comprises establishing a medical session between a primary physician and a patient; instructing during the medical session the patient from a physician station through a communication channel to engage one or more medical devices associated with a patient station; generating a request during the medical session for the one or more medical devices to take a reading from the patient that generates reading data and to transmit the reading data from the one or more medical devices to the physician station; and receiving at the physician station during the medical session the reading data from the one or more medical devices.

### BRIEF DESCRIPTION OF THE DRAWINGS

For a better understanding of embodiments of the systems and methods described herein, and to show more clearly how they may be carried into effect, reference will be made by way of example, to the accompanying drawings in which:

FIG. 1 is a block diagram illustrating the components of the system;

FIG. 2 is a block diagram illustrating the components of a physician station;

FIG. 3 is a block diagram illustrating the components of a patient station;

FIG. 4 is a block diagram illustrating the medical device gateway and one or more medical devices;

FIG. 5 is a block diagram illustrating the components of a management server;

FIG. 6A is an exemplary embodiment of the fields of a patient file database;

FIG. 6B is an exemplary embodiment of the fields of an appointment record;

FIG. 6C is an exemplary embodiment of the fields of an appointment summary record;

FIG. 7 is an exemplary embodiment of the fields of a physician database;

FIG. 8 is a flowchart illustrating the steps of a medical session method;

FIG. 9 is a flowchart illustrating the steps of a send method;

FIG. 10 is a flowchart illustrating the steps of a push method;

FIG. 11 is a sample screen shot of a physician authentication window;

FIG. 12 is a sample screen shot of a patient window;

FIG. 13 is a sample screen shot of a medical session window;

FIG. 14 is a sample screen shot of a graph of SP02 readings;

FIG. 15 is a sample screen shot of a graph of pulse readings;

FIG. 16 is a sample screen shot of a report window; and

FIG. 17 is a sample screen shot of a scheduler window.

### DETAILED DESCRIPTION OF THE INVENTION

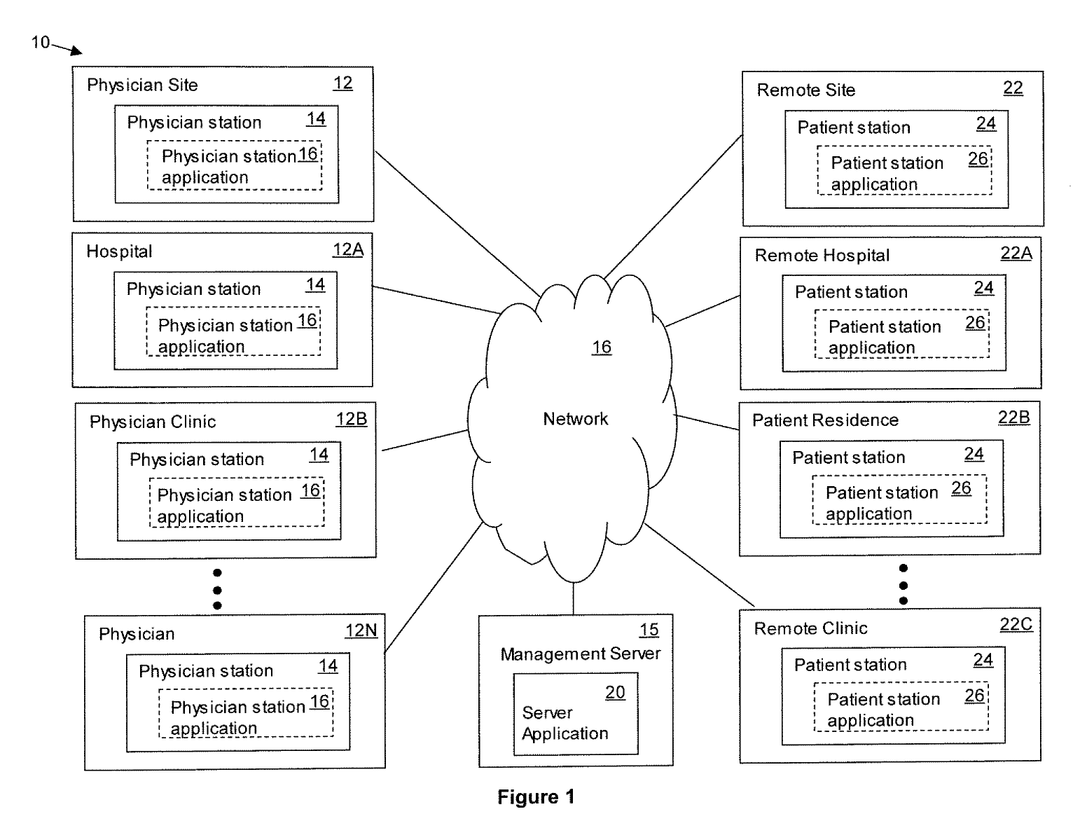

Reference is now made to FIG. 1, where the components of a remote medical system 10 are shown in an exemplary embodiment. The remote medical system 10 in an exemplary embodiment includes one or more physician sites 12, where each physician site 12 has a physician station 14. The physician stations 14 have resident upon them physician station applications 16 that are explained in further detail below. The remote medical system 10 also includes a management server 15 that has associated with it a server application 20. The remote medical system 10 further includes patient sites 22, where the patient sites 22 include patient terminals 24. The patient terminals have resident upon them, or accessible to them, patient station applications 26 and are described in further detail below. The physician stations 14 interact with patient stations 24 through a network 16.

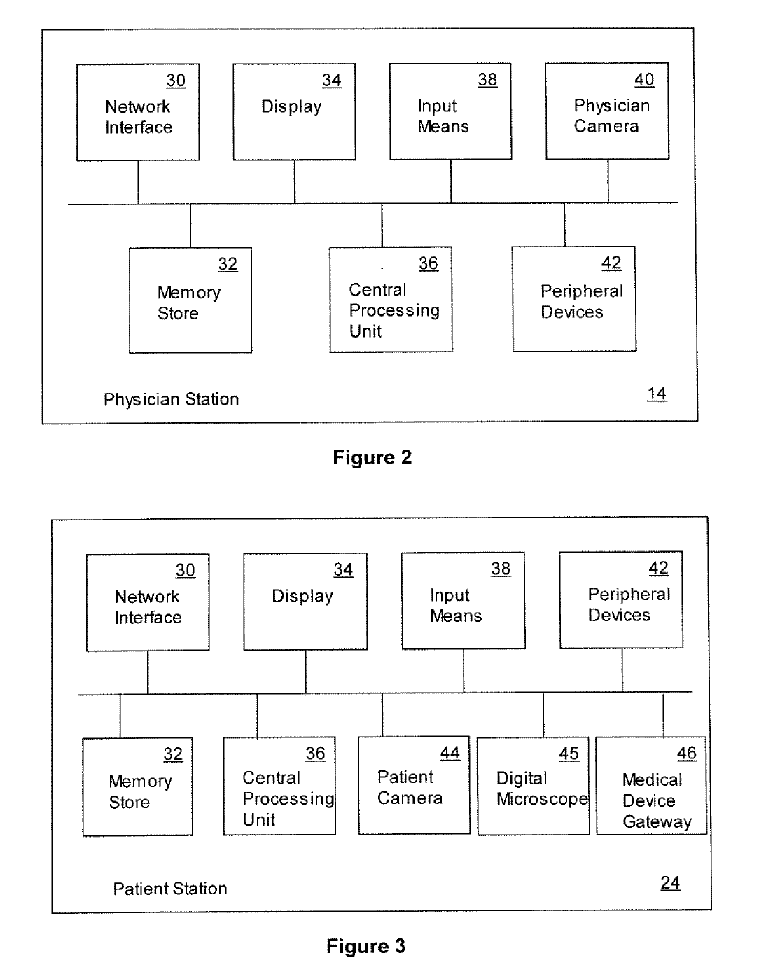

The physician sites 12 may be any site or location at which a physician station 14 is located. Sites may include, but are not limited to, hospitals 12A, physician clinics 12B, and the physician's home 12N. Any site that would allow for a physician to access a physician station 14 that connects to a network 16 may serve as a physician site 12. The physician station 14 may be any computing device that allows for a physician to interact with a patient that is in a remote location, through a computing device, and may include but is not limited to a dedicated computing device such as a kiosk or dedicated terminal, a personal computer, a laptop or slim line computer. The functions associated with a physician station 14 are further described with reference to FIG. 2 below.

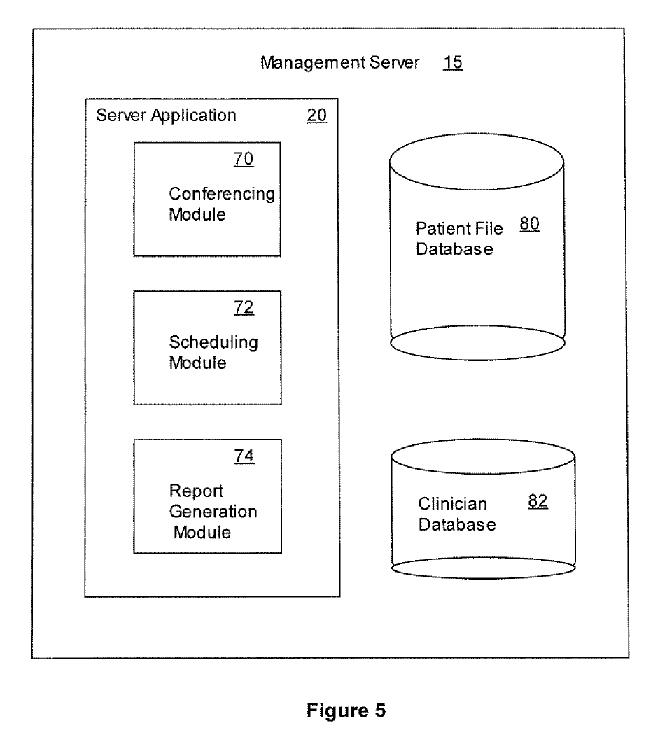

The management server 15 allows the physician station 14 to connect to a patient station 24. When the physician station and patient station 24 are connected to one another, a medical session may be conducted between a physician and patient. The medical session is any session where the physician and patient are able to interact in real-time, through either voice, video or text communication. The medical session is described in further detail below. The management server 15 stores data used to initiate and conduct a medical session, and stores medical and other data that is generated during a medical session. The server application 20 is associated with the management server 15 and is used to implement the system 10. The management server 15 and its constituent components are described in further detail below with regards to FIG. 5.

The network 16 includes multiple points of access through which data may be transmitted. The network 16 may include, but is not limited to, the publicly accessible Internet, the intranet, local area networks, or wide area networks. The network may include portions or elements of telephone lines, Ethernet connections, ISDN lines, optical-data transport links, wireless data links, wireless cellular links and/or any suitable combination of the same and/or similar elements.

The remote sites 22 may be any site or location at which a patient station 24 is located. Sites may include, but are not limited, to remote hospitals 22A, patient residences 22B, and remote medical clinics 22C. A patient located at a remote site, may take part in a medical session when the patient station 24 is connected to a physician station 14. The components associated with a patient station 24 are further described with reference to FIG. 3 below. The patient station application 26 in an exemplary embodiment, is a software application that is used to control the operation of a medical session. The patient station 14 may be any computing device that allows for a patient to interact with a physician that is in a remote location, through a computing device, and may include, but is not limited to, a dedicated computing device such as a kiosk or dedicated terminal, a personal computer, a laptop or slim line computer.

A patient and a physician, through use of the system 10 can engage in a medical session. The medical session, as is described in further detail below allows the physician and patient to interact with one another when not present in the same physical location. The physician through instructing the patient and controlling the operation of one or more medical devices, as described below, is able to engage the patient in a medical examination. The medical examination conducted when a medical session is established, allows the physician to examine the patient both visually and diagnostically, where appropriate health indicator readings are provided to the physician. Health indicator readings provide diagnostic information to physicians in regards to the patient. Health indicator readings may be received from one or more medical devices, as are described below.

Reference is now made to FIG. 2, where the constituent components of a physician station 14 are shown in an exemplary embodiment. The physician station 14, in an exemplary embodiment, has associated with it a network interface 30, a memory store 32, a display 34, a central processing unit 36, an input means 38, a physician camera 40, and peripheral devices 42.

The network interface 30 enables the respective station to connect to the communication network 16. The network interface 30 may be a conventional network card, such as an Ethernet card, wireless card, or any other means that allows for communication with the communication network 16. The memory store 32 is used to store executable programs and other information, and may include storage means such as conventional disk drives, hard drives, CD ROMS, or any other non volatile memory means. The display 34 displays the information to the user of the physician station 14 upon a monitor type device. The CPU 36 is used to execute instructions and commands that are loaded from the memory store 32. The input means 38 allows users to enter commands and information into the respective station. Physician stations 14 may have associated with them one or more input means 38, which may include, but are not limited to, any combinations of keyboards, a pointing device such as a mouse, or other means such as microphones. The physician camera 40 is a digital video camera that may be controlled by the user of the physician station 14, that is capable of capturing, storing and transmitting real-time images of the user and the surroundings of the physician station 14. Peripheral devices 42 such as printers, scanners, and other such devices may also be associated with the physician station 12.

Reference is now made to FIG. 3, where the constituent components of the patient station 24 are shown in an exemplary embodiment. In an exemplary embodiment, the physician station includes a network interface 30, a memory store 32, a display 34, a central processing unit 36, input means 38, peripheral devices 42, a patient camera 44, a digital microscope and a medical device gateway 46. The interface 30, memory store 32, display 34, central processing unit 36, input means 38 and peripheral devices 42 associated with the patient station 24, operate in a similar manner as have been described with regards to the physician station 14. The patient camera 44 is a digital camera that that may be controlled either through the physician station 14 or through the patient station 24. The patient camera 44 focuses on the patient located in proximity to the patient terminal 14, and is used to capture, and transmit in real time, images of the patient and the surroundings. The patient camera 44 may be controlled such that its tilt, rotation, zoom, and other functionality may be controlled by either user of the system through automated controls that are described in detail below. The patient camera 44 in an exemplary embodiment is a video conferencing camera with zoom, tilt and pan functionality. The digital microscope 45 may be controlled by the physician, and allows for images of the patient, and of particular areas of interest upon the patient, such as for example high-resolution images of the patient's skin, or other such features to be captured for review by the physician. The server application 20 is used to deploy updates to the patient station application 26. In an exemplary embodiment, the patient station application 26 prevents the patient from modifying any settings associated with use of the system 10. When updates that may include updated functionality, and instructions to allow for interfacing with new medical devices are provided to the patient terminal 24, the updated application is deployed through the server 15.

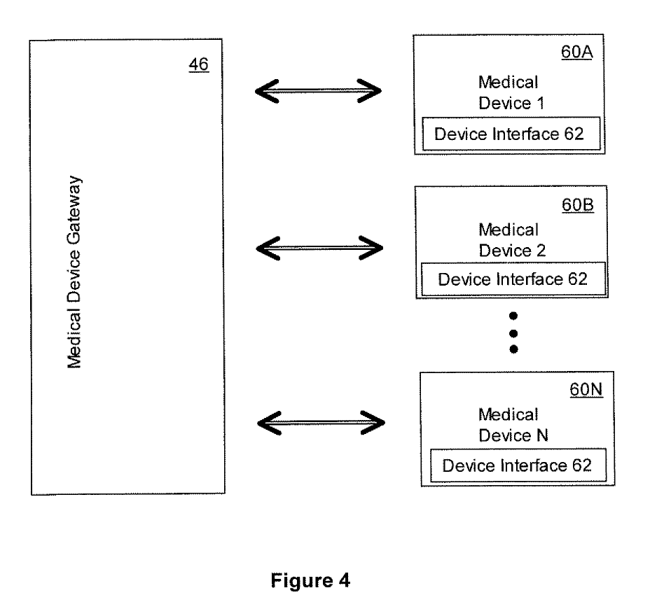

Reference is now made to FIG. 4, where the medical device gateway 46 is described in further detail below. The medical device gateway 46, in an exemplary embodiment interfaces with one or more medical devices 60. Various medial devices may used in the system 10, including but not limited to, pulse oximeters, blood pressure monitors, digital thermometers, electronic stethoscopes, ventilators, electrocardiograms, or any other such device that may be take diagnostic readings from a patient, and transmit them to a terminal. Diagnostic readings include any readings that a physician may desire when conducting a medical examination. The medical devices 60 have a device interface 62. The device gateway 46 may be used to interface with any number of medical devices 60. The device gateway 46 is able to interface with the input, output, and power components of the medical devices. The gateway device 46 in an exemplary embodiment supplies the power to the medical devices 60. The output from the medical device transmitted through the device interface 62 are received by the device gateway 46 (received as serial, binary, or any other proprietary format) and are converted to a format that allows for interfacing with the patient terminal 24. The device interface 62 may support either wired or wireless communication means. The device interface 62 is dependent upon the device, however it may include, USB communication, serial port communication, wireless means such as Bluetooth or any other suitable communication mechanism. The device interface 62 allows for data and or commands to be received by the medical device 60, and for data that represents health reading information to be transmitted from the medical device 60.

Reference is now made to FIG. 5, where the constituent components of the management server 15 are shown in an exemplary embodiment. The management server 15 has resident upon it, or accessible to it, a server application 20. The server application 20, in an exemplary embodiment comprises a conferencing module 70, a scheduling module 72, and a report generation module 74. The management server 15 also has resident upon it, or accessible to it, a patient file database 80, and a physician database 82. In an exemplary embodiment of the invention, the physician station 14 and the patient station 24 engage in a medical session communication through the Internet, where the management server 15 is accessible to both the physician station 14 and the patient station 24. The server application 20 is used to control and deploy updates to both the physician station application 16, and the patient station application 26, respectively. The management server 15 in an exemplary embodiment, is managed by an administrator of the system 10. The management server 15 may be accessed through a web site and it is not necessary that the server 15 may be accessed through the use of the physician station 14.

The server application 20 allows the physician station 14 and the patient station 24 to have their respective users engage in a medical session. The conferencing module 70 allows a medical session to be initiated and for it to take place between the physician and patient stations 14 and 24 respectively. The conferencing modules allows for video, audio and text communication to take place between the physician and patient. The scheduling module 72 controls the scheduling of medical sessions that take place between a patient and a physician, and displays appointment schedules to both the patient and physician when they access the system through their respective stations. An exemplary embodiment of the schedules that are generated and shown to the physician and patient are illustrated in detail below. The report generation module 74 is used to generate health care reports that are used to summarize details of medical sessions.

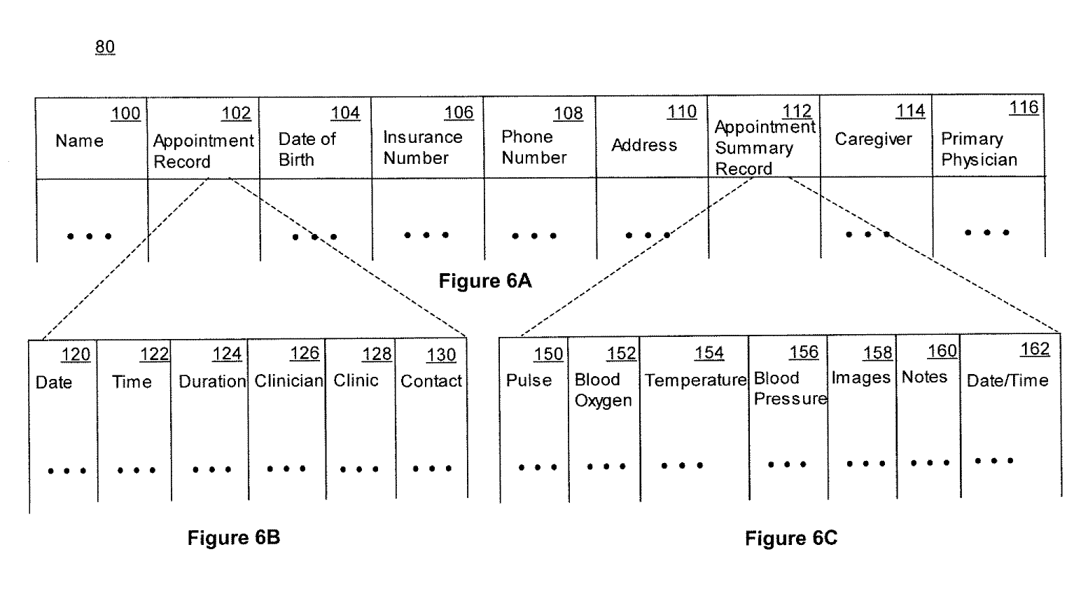

Reference is now made to FIG. 6A to 6C, where an exemplary embodiment of the fields of a patient database 80 are shown. The patient database 80 is administered by the server application 20. In an exemplary embodiment, the patient file database 80 is comprised of a name field 100, an appointment record 102, a date of birth field 104, an insurance number field 104, a phone number field 108, an address field 110, an appointment summary record 112, a caregiver field 114, and a primary physician field 116. A database record is created for each patient that uses system 10. The name field 100 stores the name of the patient. The appointment record stores details regarding the appointments a patient has for a medical session that is to be conducted with a physician. The appointment record is described in further detail in FIG. 6B. The date of birth field 104 stores the date of birth of the patient. The insurance number field 106 stores any insurance number associated with the patient that is required by the physician. The phone number field 108 stores a phone number 108 associated with the patient. The address field 110 stores the address of the patient. The appointment summary record 112 stores the health reading indicators and other information that is collected during a medical session. The appointment summary record 112 is described in further detail in FIG. 6C. The caregiver field 114 stores the name of the patient that operates the patient terminal 24 where a caregiver is needed to operate the devices and/or the terminal for the patient, as they may be unable to. The primary physician field 116 stores the name of the patient's primary physician.

Reference is now made to FIG. 6B, where the fields of the appointment record 102 are shown. The appointment record 102 records the details for each appointment the patient and physician have for a medical session. The appointment record 102, in an exemplary embodiment, comprises the following fields, a date field 120, a time field 122, a duration field 124, a physician field 124, a clinic field 128, and a contact field 130. The date field 120 stores the date of the medical session. The time field 122 stores the time the medical session is scheduled for. The duration field 124 stores the duration for which the medical session is scheduled. The physician field 126 stores both the primary physician involved in conducting the medical session, and any other physicians that were involved in the medical session or involved in consultations with the primary physician. The clinic field 128 stores the name or location of the site at which the physician is located. The contact field 130 stores the contact information associated with the physician.

Reference is now made to FIG. 6C, where an exemplary embodiment of an appointment summary record 112 are shown. The appointment summary record 112 stores, for each medical session, various health indicator readings that are taken from one or more medical devices, along with any images and physician notes that the physician has recorded during the medical session. The appointment summary record may be accessed by other physicians subsequent to the medical session, and their notes and findings may also be recorded in the summary record 112. In an exemplary embodiment, the appointment summary record 120 comprises the following fields, a pulse field 150, a blood oxygen field 152, a temperature field 154, a blood pressure field 156, an image field 158, a notes field 160, and a date/time field 162. All of the readings stored in the appointment summary record are generated based on instructions received at the respective devices that are initiated by the physician. As is described below, the physician that is engaging in a medical session with a patient is able to capture the appropriate health indicator readings, and ensure that they are not erroneous readings through reviewing the data before storing them. The pulse field 150 stores the patient's pulse as has been recorded through a medical device under control of the physician as is described in detail below. The blood oxygen field stores the blood oxygen level as measured by the medical device 60. The temperature field 154 stores the temperature recorded from a patient, as taken from a thermometer. The blood pressure field 146 stores the blood pressure readings that are taken from a patient through use of a blood pressure device. The images field 158 stores all the images the physician has captured of the patient during the medical session. The notes field 160 stores the notes the physician took regarding the medical session, and that may have been recorded by other physicians subsequent to the medical session. The notes that are made may be linked specifically to one or more instances of reading data that are stored in the patient file. The notes may include audio notes, or written notes, that are associated or linked with specific instances of reading data. Therefore, when a patient file is subsequently accessed, and instanced of reading data are reviewed, the notes that are linked to those specific instances of reading data may subsequently be reviewed as well.

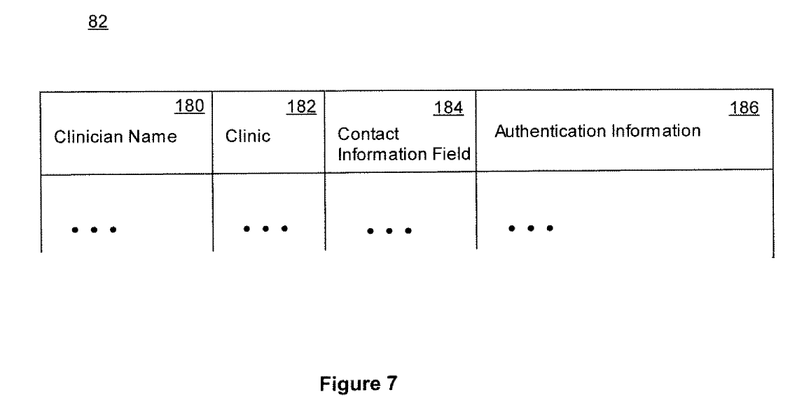

Reference is now made to FIG. 7, where a block diagram of an exemplary embodiment of the fields of a physician database 82 are shown. The physician database 82 stores information regarding the physicians who use the system 10. In an exemplary embodiment, the physician database is comprised of the physician name field 180, the clinic field 182, a contact information field 184, and an authentication information field 186. The physician name field 180 stores the name of the physician. The clinic field 182 stores the name of the clinic with which the physician is associated. The contact information field 184 stores the contact information associated with the physician, including the address, phone number and other such information. The authentication information field 186 is used to store any information that is used to authenticate a physician's access to the system 10. Authentication information may include information including, but not limited to, passwords, logins, authorization reminders, security questions, and any other such information that may be used to authenticate access to such a system 10.

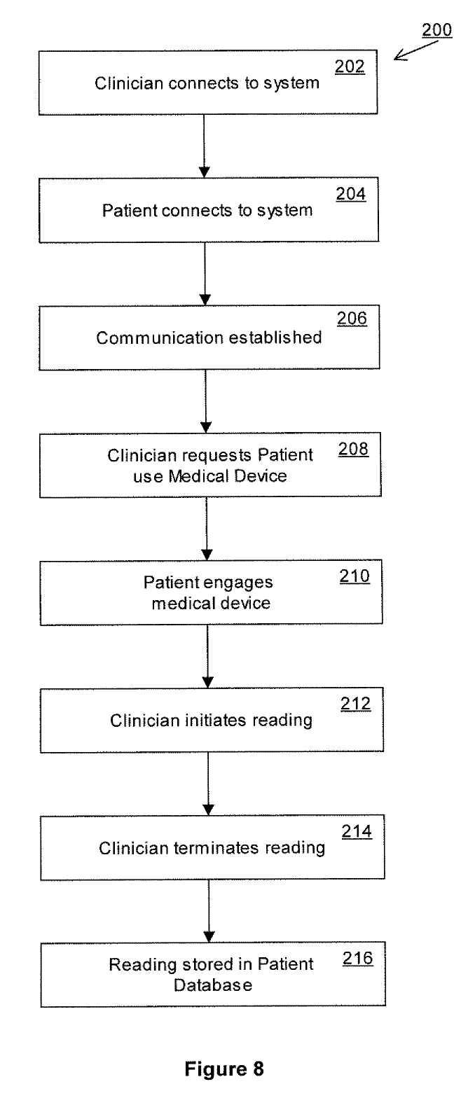

Reference is now made to FIG. 8-10 where the operation of the system 10 is further explained. In FIG. 8, a flowchart showing the general steps of a medical session method 200 is shown. The medical session may take place between a patient and one or more physicians. For every medical session, there will generally be one physician responsible for conducting the medical session and instructing the patient who is known as the primary physician. Other physicians may also be involved in the medical session either by observing the patient and monitoring the patient's health reading indicators, and/or by actively engaging the patient. A medical session is initiated based on a scheduled appointment, where the physician in an exemplary embodiment is reminded of the appointment that is scheduled. At the time of the appointment the patient is reminded that a medical session has been scheduled, and they are requested to join the session.

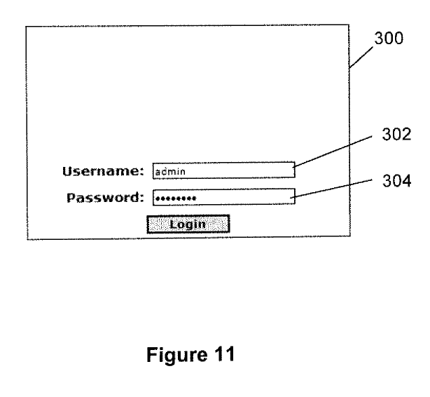

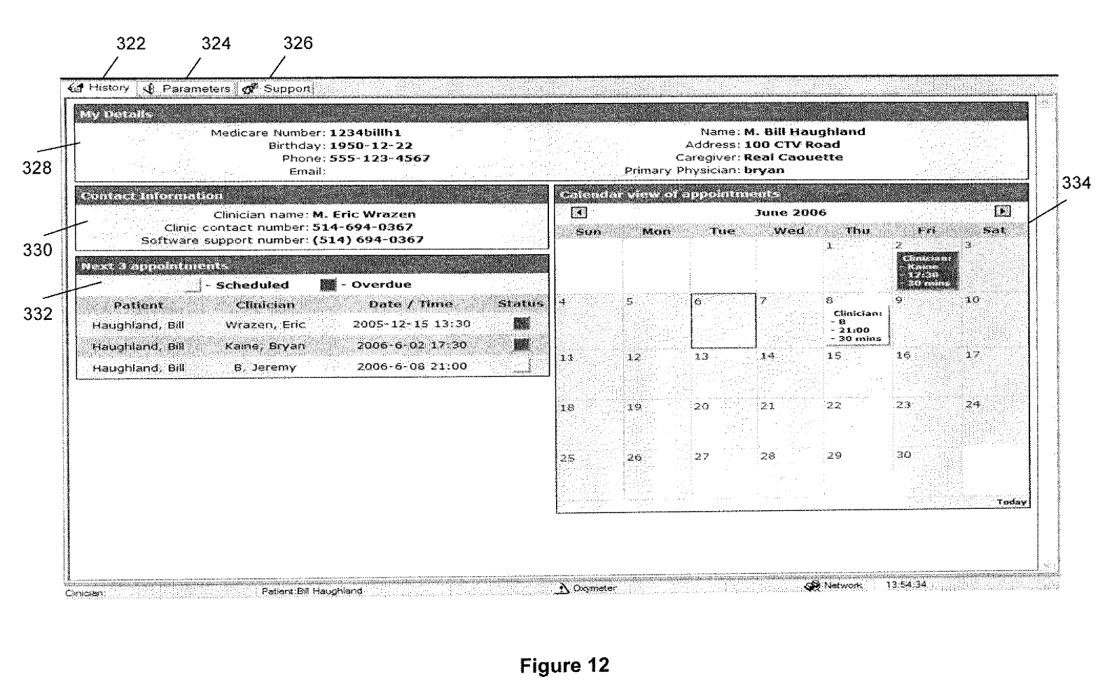

Method 200 begins at step 202, when a physician connects to the system 10. The physician connects to the system through providing authentication information to the system 10. In an exemplary embodiment, the physician provides authentication information to system through a web interface as shown in FIG. 11. Reference is now made to FIG. 11, where a sample authentication window 300 is shown. The sample authentication window 300, in an exemplary embodiment comprises a username field 302 and a password field 304. Upon entering the authentication information as required by the system 10, the management server 15, and more specifically, the server application 20, verifies that the authentication information is correct, and the physician is given access to the system 10. The physician is able to engage a patient in a medical session when a patient has accessed the system 10. At step 204, a patient through use of the patient terminal 24 accesses the system 10. Reference is made to FIG. 12, where a physician home sample screen 320 that is shown to the physician when accessing the system is shown. The physician home screen 320 provides to the patients information regarding their upcoming appointments and a schedule of appointments along with their identifying information. In an exemplary embodiment, the physician is able to control the audio and video parameters associated with receiving reading data (stethoscope audio) and video and/or audio. The physician home screen 320 in an exemplary embodiment has a details section 328, a contact section 330, an appointment section 332, and a schedule section 334. The details section 328 provides a description of the patient's information from the patient file database 80. The contact section 330 lists the contact information associated with the primary physician responsible for the patient. The appointment section 332 displays the appointments that have been scheduled for medical sessions. The appointment information is taken from the patient file database 80, where a record is kept of appointments that a patient has had, and that a patient will have. The schedule section 334 shows a calendar that is scrollable to previous and future months, and that displays the appointments that have taken place, and that are scheduled. The patient may access the system 10 in response to a request from a physician to conduct a medical session that will generally be based on a scheduled medical session, where such a request is transmitted to the patient terminal 24. In an exemplary embodiment, the patient terminal is generally associated with a specific patient. Each patient terminal in an exemplary embodiment has associated with it, a unique serial number or certificate. When a medical session has been scheduled, a virtual private network (VPN) connection is established between the respective terminals, and based on the serial number associated with the patient station 24, the patient station is authenticated. The management server 15 stores and tracks all the serial numbers associated with the respective terminals. In an exemplary embodiment, the patient terminal is always logged into the system 10. The patient, upon receiving notification that a physician is waiting to conduct a medical session, may then access the relevant screen of the system 10 that allows them to engage in a medical session. Upon being authenticated, the patient may then wait for a physician to engage in a medical session, and wait for the physician to join the medical session.

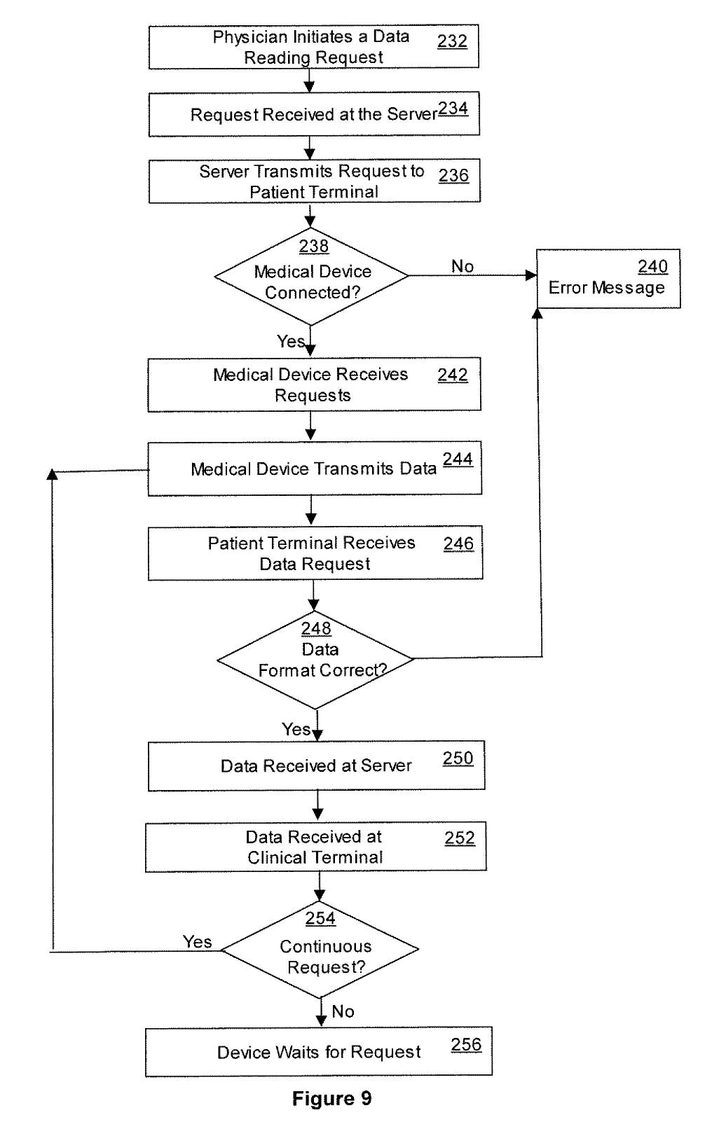

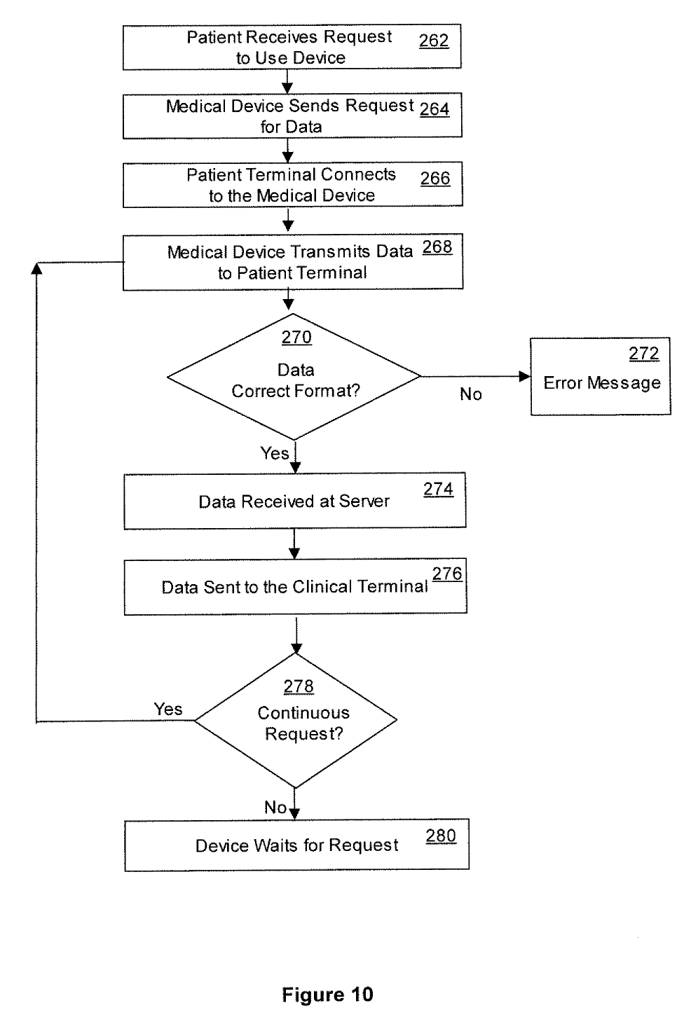

One or more physicians may join a medical session with the primary physicians permission at any time during the session. The primary physician is able to invite other physicians or medical professionals to join the session by sending them messages if they have accessed the system 10. Medical professionals may include, but are not limited to nurses, specialist physicians, primary care physicians, health care workers, mental health professionals, occupational therapists, and physiotherapists. The medical professionals may take part in an medical session through having prescheduled their participation. The medical professionals may also access the system 10 and authenticate themselves and make themselves available to participate in any medical session that is being conducted through the system 10. The primary physician is then able to chose from a list of medical professionals who are online and able to participate in the medical session, and may invite one to do so. Also, the physician is able to grant permission to one or more medical professionals to review the patient file when the medical session has been concluded. Once a medical session has been established, the respective cameras associated with the physician and patient terminals are used to capture and transmit images of their respective users. The medical session conducted between patient and physician involves the patient using one or more of the medical devices 60. At step 206, when communication between a patient and physician has been established, the patient will receive instructions from the physician. As explained below, the physician is able to instruct the patient with regards to how to use the medical devices 60. For example, instructions may include how to take a reading with the device by placing it on the appropriate part of the patient's body, and whether any features of any devices need to be activated in order for the patient to use the device upon their body. As the physician's image is captured through a physician camera 40, the image of the physician is displayed upon the patient terminal 24 in real time. The images captured by the respective cameras are transmitted through the server 15 to the respective terminals. The image and audio of both the physician and patient are captured and presented upon the respective terminals in real time. At step 208, the physician issues instructions to the patient through either visual (video or written) and audio cues. At step 210, the patient uses the medical device as instructed, under the observation of the physician, as images of patients using a medical device are transmitted to the physician station 14. The physician is able to control the patient camera 44 and is able to focus in on the use of the medical device by the patient. As is described below with respect to FIG. 9-10, the physician is able to control the medical devices as to when to take readings from the patient. The physician at step 212 is able to instruct a medical device to take a reading. The physician is able instruct the medical devices to take a reading through engaging an interface shown upon the physician station, through text commands, through voice commands, or other such suitable mechanisms. At step 214, for devices that take continuous readings, the physician is able to instruct the medical device 60 to terminate the reading. Method 200 then proceeds to step 216, where the readings taken by the medical devices are transmitted to the physician station, and then stored in the patient file database 80, and more specifically in the appointment summary record 112 that is associated with that particular medical session. In an exemplary embodiment, when the reading data is received at the physician station 14, the physician station application 16 may perform automated analysis of the reading data.

The method by which the medical device 60 receives instructions and transmits reading information to the physician station 14 is further described with reference to FIGS. 9 and 10. As described above, the device interface 62 that is associated with the medical device 60 may either be of wired or wireless means. Depending on the type of device interface 62 associated with the device, in an exemplary embodiment the device may operate in a send mode, or request mode, as described in further detail. Medical devices that rely on wireless device interfaces such as Bluetooth technology are more likely to operate on send mode. Bluetooth technologies are more likely to operate in send mode as the processing of sending data from a Bluetooth enabled device serves to notify recipients of the data that it is available to be used. The send mode operates as a method by which the respective terminals are able to recognize the Bluetooth enabled devices that are associated with the terminal. In alternative embodiments, medical devices 60 may transmit data without first receiving requests from the physician station, and may be controlled by the patient. Where the patient controls the operation of the medical devices 60, the medical devices 60 transmit all the reading data to the management server 15, that then transmits the data to the physician station 15. In such alternative embodiments, it is possible for the patient to engage the medical devices, and have all of the data transmitted to the physician station, such that the physician at the physician station may receive the data, without having to request the devices to take readings. In such alternative embodiments, the physician is able to review data and store the data to patient files, and make the appropriate notes, either in audio or written upon the patient file.

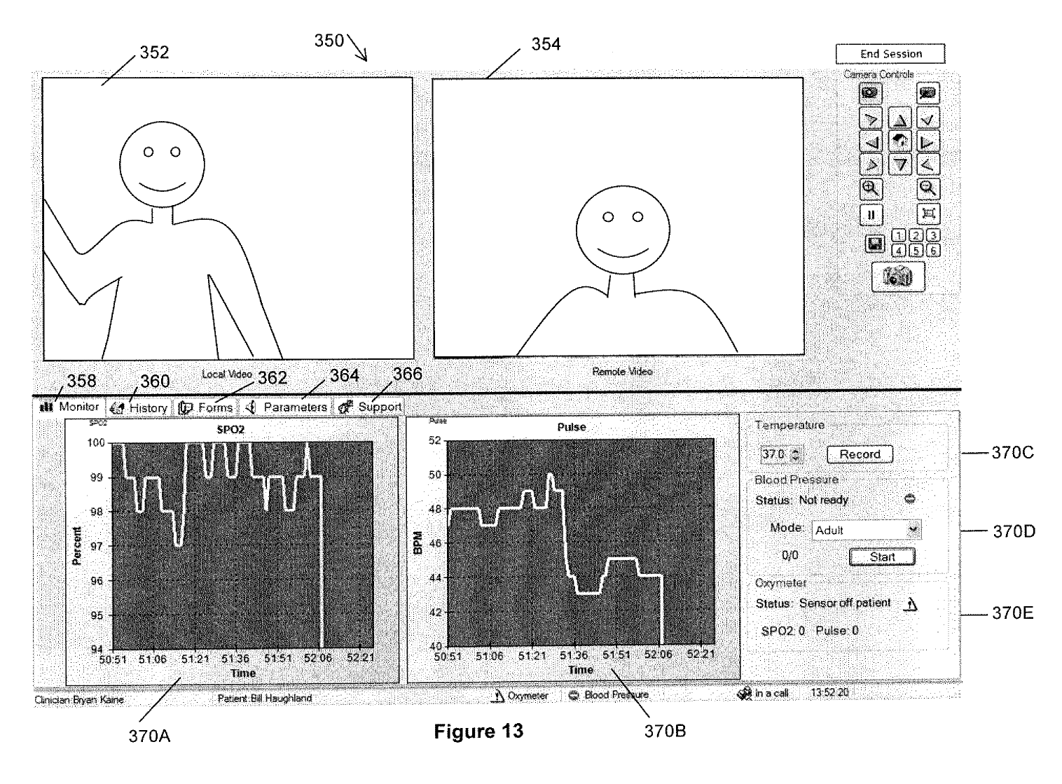

To better illustrate the functionality available to the physician, in order to describe the push and send mode, exemplary embodiments of interfaces shown to the physician and the patient are illustrated in FIG. 13-17. Reference is now made to FIG. 13, where a screenshot of a medical session window 350 is shown in one exemplary embodiment. The medical session window 350 is displayed to the physicians that are part of the medical session. The medical session window 350 in an exemplary embodiment is comprised of a physician window 352, a patient window 354, camera control 356, a monitor icon 358, a history icon 360, a forms icon 362, a parameters icon 364, a support icon 366, one or more medical device monitors 370, and device controls 372. The physician window 352 displays the image that is captured and transmitted to the patient station by the physician camera 40. The patient window 354 displays to the physician the image of the patient or the surrounding area that has been captured by the patient camera 44. The physician is able to control both the cameras through use of camera control 356. The camera control 356 allows the physician to control the operation of the patient camera 44, by zooming, tilting, rotating, and focusing the patient camera. The patient camera 44 has zoom capability that allows for it to be focused in on specific areas on the patient under the control and operation of the physician. The physician is able to focus in on specific areas upon the patient and record video and/or capture an image of the area.

The monitor icon 358, displays on the session window 350 the state of the various medical devices 60 that are associated with the patient terminal. In this example, the state of the following medical devices 60 are shown, an oxygen meter, as shown in oxygen monitor window 370A, a stethoscope as shown in the pulse window 370B, a thermometer as shown in the temperature window 370C, a blood pressure monitor as shown in the blood pressure monitor 370D, and an oxymeter as shown in the oxygen level window 370 E. As has been described above, the medical devices described with reference to session window 350 are provided for purposes of example only, as any medical device that may be used by a patient under the directions of a remotely located physician where the data from the device may be transmitted electronically may used as part of the system 10. The history icon 360, when selected, causes to be displayed the patients medical session history as taken from the patient file database 80. The parameters icon 362 allows for control of audio and video settings. The support icon 366 provides contact information regarding who to contact if any support is needed when using the system 10. Depending on the type of medical device 60 and commands issued by the physician, the constant output of data generated by the medical devices may be transmitted for the physician to review. In this example, the physician receives real time updates of the readings that are taken by the respective medical devices 60 that are engaged by the patient. Readings taken by the medical devices 60 may be displayed upon the session window 350 in a graph format as is done in SPOS window 370A and pulse window 370 B. Other readings including, but not limited to, the temperature reading as shown in the temperature window 370C, the blood pressure reading as shown in the blood pressure window 37D, and the oxymetry reading as shown in the oxymeter window 370 E, are shown in windows where readings taken at one instance of time are shown. The SP02 window 370A and the pulse window 370 B provide real time continuous readings that are being taken by their respective devices. By allowing the physician to monitor the readings in real time, the physician is able to determine whether the patient has engaged the device correctly, and to be able to get a more accurate understanding of the health conditions facing the patient.

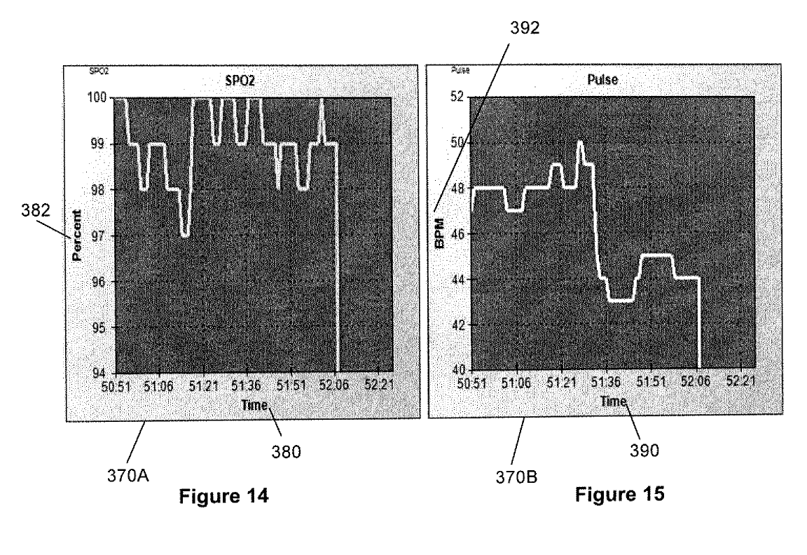

Reference is now made to FIGS. 14 and 15, where a graph window of the SPOS window 370A and the pulse window 370B are explained in greater detail. The medical devices 60 that continuously transmit data, may have their readings displayed as graphs in a session window. By allowing the physician to constantly monitor the readings taken by such devices, the physician is able to determine whether the device is being used properly (i.e. if readings that are generated are not reflective of readings that may be received from a patient, the physician may ask the patient to adjust the use of the device 60 upon their body). Also, by having real time access to continuous readings, the readings are stored for further review. In the SP02 window 370A a graph of SP02 percentage 382 vs. time 380 is displayed. In the pulse window 370B, the pulse readings 392 are graphed vs. time 390.

Reference is now made again to FIG. 9, where a flowchart illustrating the steps of a send method 230 is shown in an exemplary embodiment. Upon engaging the medical device 60 on the patient's body, the medical device 60 may receive requests for readings to be taken and transmits the data to the physician terminal through either a send method or a push method. The steps of the send method, in an exemplary embodiment are described here. The send method 230 is initiated when a physician initiates a request for a medical device 60 to take a reading at step 232. The physician may initiate a reading request by making use of the functionality that is provided upon one of the session windows 350. For example, in session window 350, the physician is provided with a start button in the blood pressure monitoring window 374, that when engaged, generates a request for a blood pressure reading. In the system 10, all of the medical devices 60 that are connected to the device gateway 46 may have readings requested through the physician's use of the interfaces provided upon the physician station 14. By being able to visually observe the location of the device upon the body of the patient through use of the respective cameras, the physician when confident that the patient has engaged the device in a proper manner, may request that a reading be taken from the medical device 60. Method 230 then proceeds to step 234, where the reading request that has been encrypted is sent to the server 15. As the medical session is established through a VPN connection, in an exemplary embodiment secure socket layer (SSL) technology is used to encrypt the data. Method 230 then proceeds to step 236, where the server 15 transmit the request to the patient terminal 24. Upon receiving the request at the patient terminal 24, the medical devices 60 that are being request to take readings are identified. Method 230 then proceeds to step 238, where a check is performed to determine whether the device 60 is connected to the gateway 46 either through wired or wireless means. For example, it may be the case that the device 60 has its power turned off, or there may be errors that prevent it from being able to transmit data through its respective device interface 62. The check performed at step 238 determines whether the device 60 is able to transmits readings it takes. If it is determined at step 238 that the requested medical device 60 is not able to transmit data correctly, an error message is generated at step 240. The error message is displayed to both the physician and the patient upon their respective terminals. The error message allows the patient and physician to attempt to diagnose why the device 60 is not able to transmit readings. If at step 238, it is determined that the device 60 is able to transmit readings, method 230 proceeds to step to step 242. At step 242, the medical device 60 receives requests to initiate a reading from the patient terminal, and more specifically from the patient terminal application 26. At this point, the device is engaging the appropriate area upon the body of the patient, and readings may be taken through using the device at that particular area. At step 244, the medical device begins to generate readings and transmits the reading data. Method 230 then proceeds to step 246 where the patient terminal 24 receives the data that is being transmitted from the device 60 that is transmitted through the device gateway 46. Method 230 then proceeds to step 248, where a check is performed to determine whether the format of the data that has been sent from the medical device is the correct format. If it is determined that the format is not correct, an error message is generated. The format may not be correct where the data may have been corrupted in transmission. If it is determined that the data format is correct, method 230 proceeds to step 250. At step 250, the data is transmitted to and received at the server 15. The data that is transmitted to the server 15 is transmitted securely, as it is encrypted. Method 230 then proceeds to step 252, where the data is received at the physician terminal 14, where it is decrypted, and is displayed upon the physician terminal, through one of the respective session windows. Method 230 then proceeds to step 254, where a check is performed to determine whether the device is to transmit data continuously. For example, some devices such as oxygen monitors, and devices that determine a patient's pulse, are to transmit data continuously so that the readings may be monitored over a period of time. If the device 60 is to transmit data continuously, the data that is being transmitted from the device 60 continues to be received by the patient terminal at step 246, and after the appropriate determinations and encryption are performed, it is transmitted to the server 15. If it is determined at step 254, that the device 60 is not required to transmit data continuously, the device 60 is instructed to wait for another request to take a reading. When the device 60 is sending data continuously, it will do so until the physician issues a termination request, where the physician sends a request to the device to terminate the readings. Examples of such devices that wait for termination readings include blood pressure monitors.

The medical devices 60 that are connected to the patient terminal may operate either in send mode or push mode. Reference is now made again to FIG. 10, where a flowchart illustrating the steps of a send method 260 in an exemplary embodiment are shown. Method 260 begins at step 262, where a physician, based on the functionality that is provided initiates a request for a reading to be taken. Method 260 then proceeds to step 264, where the medical device 60 after having received the request to transmit readings, sends a request to the patient terminal. It should be noted, that not all medical devices 60 need to be instructed to initiate readings. For example, as part of the system 10, a weighing scale that is wireless enabled may be used, where upon stepping on the scale, the scale transmits the weight reading to the device gateway 46. At step 266, the patient terminal connects to the medical device through the device gateway 46. Method 260 then proceeds to step 268, where the device 60 begins to generate readings and transmits the data to the patient terminal 24. At step 270, a check is performed to determine whether the data transmitted from the medical device 60 is in the correct format, as the data may have been corrupted in transmission. If it is determined at step 270, that the data is not in the correct format, an error message is generated and method 260 proceeds to step 272, where a message is displayed upon the patient terminal 14. If it is determined at step 270, that the data format is correct, method 260 proceeds to step 274. At step 274, the data is transmitted from the patient terminal 24, to the server 15, in an encrypted manner. Method 260 then proceeds to step 276, where the data is received at the physician terminal 276. Upon receipt of the data at the physician terminal, a decryption process is undertaken, and the readings are then displayed to the physician upon the respective terminal 14. A check is performed at step 278 to determine whether the device is required to transmit the data continuously. If it is determined at step 278 that the device is required to transmit the data continuously, the device 60 continues to transmit data to the patient terminal that is then transmitted through the server 15 and sent to the physician terminal 14. If it is determined at step 278 the device 60 does not transmit data continuously, method 260 proceeds to step 280 where the device is instructed to wait for another request to transmit data. Where the device transmits data continuously, the device 60 will do so until a termination request is sent from the physician.

During a medical session, where the physician has received various health indicator readings, the physician may save the readings to the patient file stored in the patient file database. As well as being able to generate images of the patient and store the images of the patient in the database, the physician is able to store any written notes associated with the patient, as well as audio records that may be made electronically of the conversation that has taken place between the patient and physician, or of the physician's audio notes. Other physicians that are taking part in a medical session are also able to have their notes (audio and written) saved in the patient file. Where a patient file is accessed after the termination of a medical session, approved physicians are able to add notes to the file, and the notes will be recorded in the file and will specify the physician that has made the notes. This allows specialist physicians to review a patients file after the completion of a medical session, and for the physician to provide their findings and record their findings within the patient file.

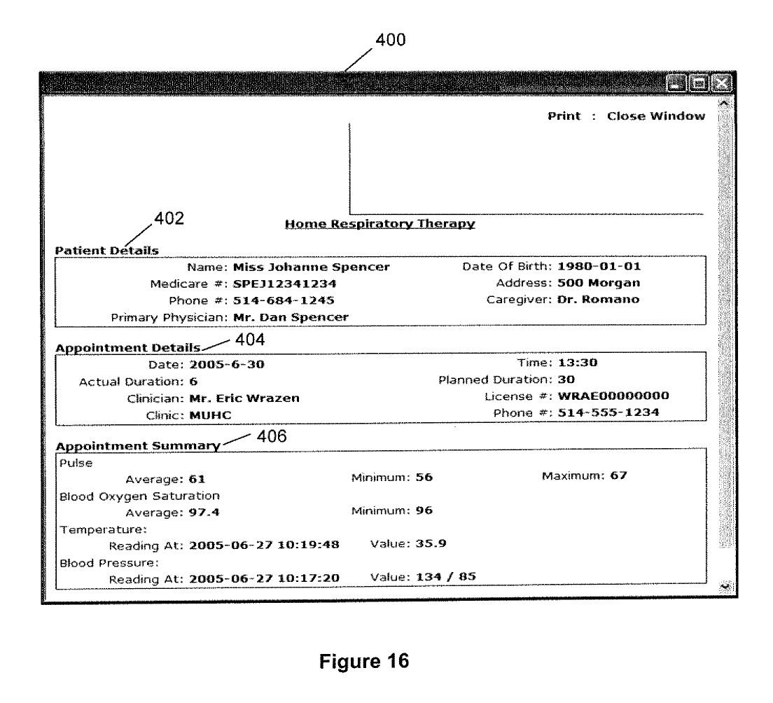

Upon conclusion of the medical session, the report generation module 74 associated with the server application 20, generates a report that summarizes the medical session. Reference is now made to FIG. 16, where an exemplary embodiment of a report that is generated by the report generation module is shown. The report generation window 400, is displayed upon the physician and patient terminals, and summarizes the medical session the physician and patient have participated in. In an exemplary embodiment, the report generation window 400 comprises a patient details section 402, an appointment details section 404, and an appointment summary 406. The patient details section 402 lists the information that is contained with the patient's file as accessed from the patient file database 80. The appointment details section 404 summarizes the date, time and contact information of the primary physician that has conducted the medical session. The appointment summary section 406 summarizes the health reading indicators that were requested and recorded during the session and displays them. The patient may print this report for their records.

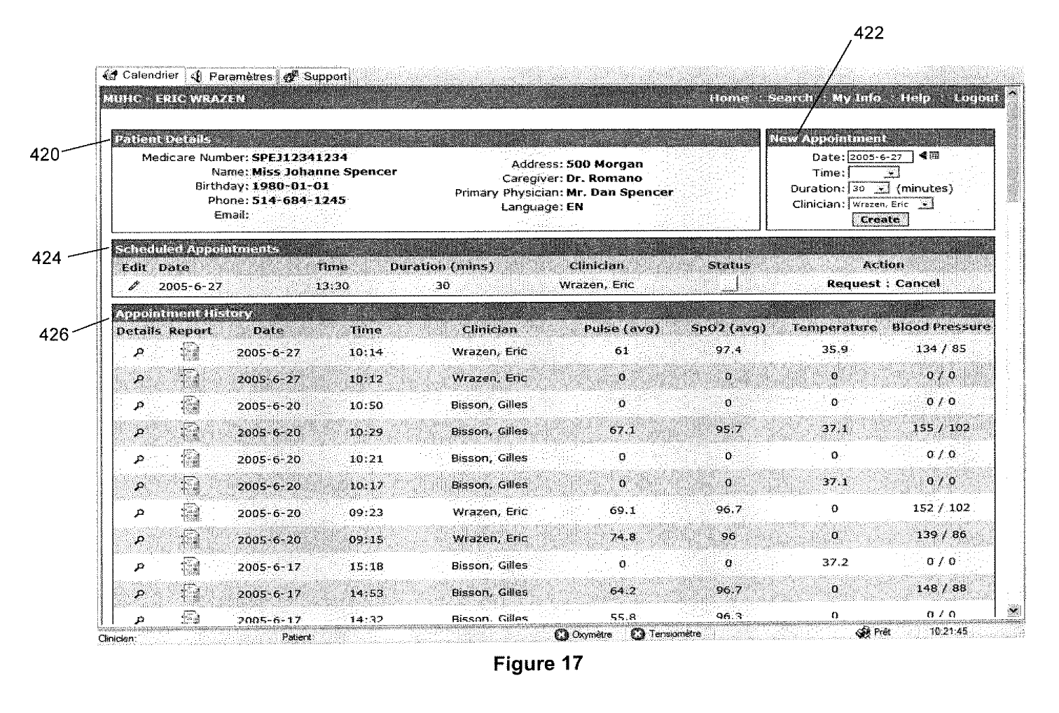

The physician and patient upon conclusion of their medical session, are presented with the option of scheduling their next medical session. Reference is made now made to FIG. 17, where an exemplary embodiment of an appointment scheduler window 420 is shown. The appointment scheduler 420 allows the physician and patient to schedule the next medical session while they are in communication with one another. The schedule window 420, in an exemplary embodiment comprises a new appointment window 422, a patient details section 424, a scheduled appointment section 424 and an appointment history window 428. The new appointment window 422 allows the physician to select a time and date, and the primary physician that will conduct the medical session.

The present invention has been described with regard to exemplary embodiments. However, it will be obvious to persons skilled in the art that a number of variants and modifications can be made without departing from the scope of the invention as described herein.

1. A method for conducting examinations of patients at remote locations, the method comprising:
a) establishing a medical session between a primary physician and a patient;
b) instructing during the medical session the patient from a physician station through a communication channel to engage one or more medical devices associated with a patient station;
c) generating a request during the medical session for the one or more medical devices to take a reading from the patient that generates reading data and to transmit the reading data from the one or more medical devices to the physician station; and
d) receiving at the physician station during the medical session the reading data from the one or more medical devices.

2. The method of claim 1, wherein the reading data is stored in a patient file.

3. The method of claim 2, wherein the patient file includes notes made by the physician.

4. The method of claim 3, wherein the notes may be linked to reading data received from the one or more medical devices.

5. The method of claim 2, wherein the patient file is stored upon a management server.

6. The method of claim 2, wherein the patient file stores one or more images taken of the patient.

7. The method of claim 3, wherein one or more medical professionals are granted permission to review the patient file.

8. The method of claim 7, wherein the one or more medical professionals include notes in the patient file.

9. The method of claim 1, wherein the reading data is transmitted to the physician station through a management server.

10. The method of claim 1, wherein the request for one or more medical devices to take the reading, is generated by engaging an interface presented at the physician station.

11. The method of claim 1, wherein the one or more medical devices are selected from the group comprising pulse oxymeters, blood pressure monitors, digital thermometers, electronic stethoscopes, ventilators, digital microscopes, weighing scales, and electrocardiograms.

12. The method of claim 1, wherein the communication channel allows for voice communication.

13. The method of claim 1, wherein the communication channel allows for text communication.

14. The method of claim 1, wherein the communication channel allows for real-time video communication.

15. The method of claim 1, wherein the physician controls the operation of a patient camera associated with the patient station.

16. The method of claim 1, the method further comprising granting permission to one or more medical professionals to participate in the medical session.

17. The method of claim 16, wherein the one or more medical professionals have made themselves available online to participate in any medical session.

18. The method of claim 16, wherein the one or more medical professionals are located in locations other than those of the primary physician and the patient.

19. The method of claim 16, wherein the one or more medical professionals record notes in a patient file.

20. The method of claim 1, wherein during the medical session the primary physician and the patient schedule a next medical session.

21. The method of claim 20, wherein the primary physician and the patient view an online calendar to schedule a next medical session.

22. The method of claim 1, further comprising the step of undertaking automated analysis of the reading data received from the one or more medical devices.

23. The method of claim 22, wherein the automated analysis is based on predefined rules used in the automated analysis.

24. The method of claim 1, wherein the medical devices are connected to a device gateway.
